(19) United States
(12) Patent Application Publication
(10) Pub. No.: US 2012/0010484 A1
(43) Pub. Date: Jan. 12, 2012

(54) PHOTOBIOLOGICAL MEASURING DEVICE AND ANALYZING METHOD

(75) Inventor: Akihiro Ishikawa, Kyoto (JP)

(73) Assignee: Shimadzu Corporation, Kyoto (JP)

(21) Appl. No.: 13/256,287

(22) PCT Filed: Jan. 12, 2010
(86) PCT No.: PCT/JP2010/050205

§ 371 Date: Sep. 13, 2011

(30) Foreign Application Priority Data
Mar. 19, 2009 (JP) 2009-068351

(51) Int. Cl. A61B 5/1455

(52) U.S. Cl. 600/310

(57) ABSTRACT

A photobiological measuring device has a computing unit generating an unwanted component removal observation signal by removing the signal corresponding to the unwanted component from an observation signal. The computing unit is provided with: a mixing matrix making unit for separating observation signals into the products of a mixing matrix and independent component signals through independent component analysis; a power spectrum computing unit generating transformed independent component signals, which are functions of frequency and intensity, by Fourier transforming the independent component signals, which are functions of time and intensity, and computing power spectra in predetermined frequency bands of transformed independent component signals; and an unwanted component signal determining unit detecting the independent component signal corresponding to the unwanted component from among the independent component signals by comparing the power spectrum in a predetermined frequency band of each transformed independent component signal with a threshold.

### CROSS REFERENCE TO PRIOR APPLICATIONS

This application is a U.S. National Phase application under 35 U.S.C. §371 of International Application No. PCT/JP2010/050205, filed on Jan. 12, 2010 and claims benefit of priority to Japanese Patent Application No. 2009-068351, filed on Mar. 19, 2009. The International Application was published in Japanese on Sep. 23, 2010 as WO 2010/106826 A1 under PCT Article 21(2). All of these applications are herein incorporated by reference.

### TECHNICAL FIELD

The present invention relates to a photobiological measuring device and analyzing method for acquiring an observation signal which indicates variations chronologically in terms of a portion to be measured using light, and in particular to an optical encephalographic device for measuring the function of a portion of the brain in a non-invasive manner using near-infrared rays (fNIRS: functional near-infrared spectroscope), or an oxygen monitor for monitoring the amount of oxygen consumption in the portion to be measured in a living body.

### BACKGROUND

A photobiological measuring method for measuring the inside of a living body easily and in a non-invasive manner through the fact that the concentration of hemoglobin corresponds to the oxygen metabolizing function inside the living body has been known. According to this photobiological measuring method, the concentration of hemoglobin is found from the amount of light that transmits through a living body when the living body is irradiated with light of which the wavelength ranges from the visible light region to the near-infrared region. Furthermore, hemoglobin combines with oxygen to form oxyhemoglobin or separates from oxygen to form deoxyhemoglobin. It is also known that within a brain, oxygen is supplied to the activated portions through the blood flow redistribution function so that the concentration of oxyhemoglobin increases as a result of the combination with oxygen. Therefore, brain activity can be monitored by measuring the concentration of oxyhemoglobin. Oxyhemoglobin and deoxyhemoglobin have different spectral absorbing properties in the region ranging from visible light to near-infrared rays, and therefore the concentration of oxyhemoglobin and the concentration of deoxyhemoglobin can be respectively found using near-infrared rays having two different wavelengths (for example, 780 nm and 850 nm).

Thus, a light measuring device with a holder (light transmitting/receiving unit) having a number of light transmitting probes and a number of light receiving probes has been developed (see Japanese Unexamined Patent Publication 2006-109964). The light measuring device allows the brain to be irradiated with near-infrared rays by means of the light transmitting probes provided on the surface of the scalp of a subject, and at the same time detects the amount of near-infrared rays emitted from the brain by means of the light receiving probes provided on the surface of the scalp. In addition, the light transmitting probes and the light receiving probes are inserted into through holes provided in the holder so that the distances (channels) between the light transmitting probes and the light receiving probes are constant, and the light detection signal (amount of light) can be gained from a number of portions of the brain at a certain depth from the surface of the scalp.

FIG. 2 is a plan diagram showing the positional relationships between 16 light transmitting probes and 16 light receiving probes in a holder. The light transmitting probes 12 and the light receiving probes 13 are arranged in a tetragonal lattice structure so as to alternate both in the row and column directions in the holder 11. The holder 11 is designed by taking the distance between the scalp and the brain into consideration, and when the subject is an adult, the distance (channel) between the light transmitting probes 12 and the light receiving probes 13 is 30 mm. When the channel is 30 mm, it is possible for the light detection signal to be gained from the location at a depth of 15 mm to 20 mm from the middle point in the channel. That is to say, the location at a depth of 15 mm to 20 mm from the surface of the scalp corresponds approximately to a portion on the surface of the brain, and thus 52 light detection signals indicating chronological variations can be gained in terms of 52 portions on the surface of the brain (#1 to #52). Though light emitted from the light transmitting probes 12 can be detected by distant light receiving probes 13 other than the adjacent light receiving probes 13, it is assumed that in order to simplify the description, only the adjacent light receiving probes 13 can detect the light.

On the basis of the thus-gained 52 light detection signals, the concentration of oxyhemoglobin (observation signals) in 52 portions on the surface of the brain (#1 to #52) Xₙ(t) (n=1, 2 . . . 52), the concentration of deoxyhemoglobin (observation signals) and the total concentration of hemoglobin (observation signal), which is calculated from these, can be found. FIG. 3 is a diagram showing a monitor screen of the photobiological measuring device displaying the 52 concentrations of oxyhemoglobin Xₙ(t) (#1 to #52). Here, the longitudinal axis for each observation signal Xₙ(t) indicates the concentration of oxyhemoglobin and the lateral axis indicates time t.

Incidentally, the 52 displayed observation signals Xₙ(t) in FIG. 3 consist of overlapping signals on the basis of fluctuations in the blood flow through the skin and the heart rate as well as variations in pulsation and respiration, in addition to signals on the basis of the blood flow variations according to the brain functions.

Thus, the observer, such as a doctor, makes a sharp distinction visually between the signal on the basis of the blood flow according to the brain function in the observation signal Xₙ(t) and other signals so that the observer can easily diagnose whether or not there is a condition, such as cerebral ischemia. For example, only the signal that is in sync with the brain's task is handled as a brain function signal, or a signal that is difficult to be regarded as a physiological change within the brain is handled as an artifact. Furthermore, random noise is removed through an integration process with a great number of repeated measurements.

According to another method for easily diagnosing whether or not there is a condition such as cerebral ischemia, the observation signal Xₙ(t) is statistically analyzed using a linear model (GLM), and the result is compared with the reference observation signal so that the similarity between the reference observation signal and the observation signal Xₙ(t) as well as the evaluation of statistical significance (value P) can be calculated as the statistical result (see Japanese Unexamined Patent Publication 2003-265442).

When the brain function of a subject is measured with a movement such as walking provided as a task is measured visually as described above, the heart rate changes and the signal on the basis of the change in the blood flow in the skin is also in sync with the task. Therefore, when only the signal that is in sync with the task is handled as the brain function signal, in some cases it cannot be precisely diagnosed whether or not there is a condition such as cerebral ischemia.

Though the statistical analysis as described above makes it possible to find the similarity and the evaluation (value P) concerning the observation signal Xₙ(t), in some cases it cannot be precisely diagnosed whether or not there is a condition such as cerebral ischemia because signals on the basis of fluctuations in the blood flow in the skin and the heart rate as well as variations in pulsation and respiration in addition to the signals on the basis of the blood flow according to the brain functions keep overlapping the observation signal Xₙ(t).

Thus, an example provides a photobiological measuring device and analyzing method according to which the signal corresponding to an unwanted component can be removed from the observation signal.

### SUMMARY

The photobiological measuring device according to the present example, has: a light transmitting/receiving unit having a number of light transmitting probes to be provided on the surface of the skin of a subject and a number of light receiving probes to be provided on the surface of the skin; a light transmitting/receiving unit control unit for acquiring an observation signal indicating chronological variations concerning a portion to be measured in a subject by allowing the above described light transmitting probes to irradiate the surface of the skin with light while controlling the above described light receiving probes in order to detect light emitted from the surface of the skin; and a computing unit for generating an unwanted component removal observation signal by removing the signal corresponding to the unwanted component from the above described observation signal, wherein the above described computing unit has: a mixing matrix making unit for separating observation signals into the products of a mixing matrix and independent component signals through independent component analysis; a power spectrum computing unit for generating transformed independent component signals, which are functions of frequency and intensity, by Fourier transforming the independent component signals, which are functions of time and intensity, and thus computing power spectra in predetermined frequency bands of the transformed independent component signals; and an unwanted component signal determining unit for detecting the independent component signal corresponding to the unwanted component from among the independent component signals by comparing the power spectrum in a predetermined frequency band of each transformed independent component signal with a threshold.

Here, the “observation signal” may be a light detection signal that is detected by light receiving probes or the concentration of oxyhemoglobin, the concentration of deoxyhemoglobin or the total concentration of hemoglobin, which are calculated from the light detection signal.

In addition, the “signal corresponding to an unwanted component” can refer to a signal other than the signal on the basis of the blood flow according to the brain function and is the signal on the basis of the blood flow in the skin, the signal on the basis of the fluctuations in the heart rate, and the signal on the basis of pulsation or respiration, for example.

Furthermore, the “predetermined frequency band” refers to any frequency band that can be set in advance, and the frequency band indicating the blood flow in the skin (0.03 Hz to 0.15 Hz), the frequency band indicating the fluctuations in the heart rate (0.15 Hz to 0.5 Hz), and the frequency band indicating pulsation or respiration (0.8 Hz to 2.0 Hz) can be set as the “predetermined frequency band,” for example.

In the photobiological measuring device according to the present example, the light transmitting/receiving control unit allows the light transmitting probes to irradiate the surface of the skin with light and control the light receiving probes in order to detect light emitted from the surface of the skin, and thus N observation signals Xₙ(t) concerning N portions to be measured can be acquired. Here, the observation signals Xₙ(t) consist of overlapping signals on the basis of the fluctuations in the blood flow in the skin and the heart rate as well as variations in pulsation and respiration, in addition to signals on the basis of the blood flow according to the brain functions.

The computing unit removes the signal corresponding to an unwanted component from the observation signals Xₙ(t). First, as shown by the following formula (1), the mixing matrix making unit separates the N observation signals Xₙ(t) into the products of an N×N mixing matrix and N independent component signals Sₙ(t) through independent component analysis (ICA).

$$\begin{matrix}[ Formula 1 ] & \\ {( \begin{matrix}{X}_{1} ( t ) \\ {X}_{2} ( t ) \\ ⋮ \\ {X}_{n} ( t )\end{matrix} )}^{Observation Signal} = {( \begin{matrix}{a}_{11} & {a}_{12} & … & {a}_{1 n} \\ {a}_{21} & {a}_{22} & … & {a}_{2 n} \\ … & … & … & … \\ {a}_{n 1} & {a}_{n 2} & … & {a}_{nn}\end{matrix} )}^{Mixing Matrix} {( \begin{matrix}{S}_{1} ( t ) \\ {S}_{2} ( t ) \\ ⋮ \\ {S}_{n} ( t )\end{matrix} )}^{\\ {Component Signal}^{Independent}} & ( 1 )\end{matrix}$$

Here, the column vector in the mixing matrix indicates the weight of a certain independent component signal Sₙ(t) in a portion to be measured. That is to say, the observation signals Xₙ(t) are a linear combination of the N independent component signals Sₙ(t) from independent sources for generating signals with each element in the mixing matrix as a weight coefficient.

In the case where there is a signal source for a signal on the basis of the blood flow in the skin, which is irrelevant of the signal on the basis of the blood flow according to the brain function, it is possible for some of the N independent component signals Sₙ(t) to be the signal on the basis of the blood flow in the skin from the signal source.

It is generally known that signals on the basis of the blood flow in the skin appear in a predetermined frequency band λ₁. Therefore, in order to find out the independent component signal Sₙ(t) corresponding to an unwanted component from among the N independent component signals Sₙ(t), the power spectrum computing unit Fourier transforms the N independent component signals Sₙ(t) so as to generate N transformed independent component signals Sₙ(λ). Thus, the power spectrum computing unit calculates the power spectrum Sₙ(λ₁) of the predetermined frequency band λ₁ in the N transformed independent component signal Sₙ(λ).

Next, the unwanted component signal determining unit compares the N power spectra Sₙ(λ₁) with the respective thresholds T so as to find the independent component signal Sₙ(t) corresponding to an unwanted component from among the N independent component signals Sₙ(t). In the case where the power spectrum S₁(λ₁) is not less than the threshold T, for example, the independent component signal S₁(t) is determined to be the signal corresponding to an unwanted component. Meanwhile, in the case where the power spectrum S₁(λ₁) is less than the threshold T, the independent component signal S₁(t) is determined to be a signal on the basis of the blood flow according to the brain function. Here, the number of signals that are determined to correspond to an unwanted component is not necessarily one, but may be plural.

### Effects of the Invention

As described above, the photobiological measuring device can remove the signals corresponding to an unwanted component from the observation signals Xₙ(t).

### Other Means for Solving Problem and Effects of the Invention

In addition, in the photobiological measuring device according to the present example, the above described computing unit may have: an unwanted component removal mixing matrix making unit for making an unwanted component removal mixing matrix in which 0 is substituted for the column vector corresponding to an unwanted component in the above described mixing matrix on the basis of the independent component signal corresponding to an unwanted component found in the above described unwanted component signal determining unit; and an unwanted component removal observation signal generating unit for generating unwanted component removal observation signals by multiplying an unwanted component removal mixing matrix with independent component signals.

In the photobiological measuring device according to the present example, the unwanted component removal mixing matrix making unit first makes an N×N unwanted component removal mixing matrix by substituting 0 for the column vector corresponding to an unwanted component in the N×N mixing matrix on the basis of the independent component signal Sₙ(t) corresponding to an unwanted component found in the unwanted component signal determining unit. For example, when the power spectrum S₁(λ₁) is not less than the threshold T, an unwanted component removal mixing matrix is made by substituting 0 for the first column vector.

Next, as shown in the following formula (2), the unwanted component removal observation signal generating unit multiplies the N×N unwanted component removal mixing matrix by the N independent component signals Sₙ(t) in order to generate N unwanted component removal observation signals Xₙ′(t).

$$\begin{matrix}[ Formula 2 ] & \\ {( \begin{matrix}{X}_{1}^{′} ( t ) \\ {X}_{2}^{′} ( t ) \\ ⋮ \\ {X}_{n}^{′} ( t )\end{matrix} )}^{{Removal Observation Signal}^{Unwanted Component}} = {( \begin{matrix}0 & {a}_{12} & … & {a}_{1 n} \\ 0 & {a}_{22} & … & {a}_{2 n} \\ 0 & … & … & … \\ 0 & {a}_{n 2} & … & {a}_{nn}\end{matrix} )}^{{Removal Mixing Matrix}^{Unwanted Component}} {( \begin{matrix}{S}_{1} ( t ) \\ {S}_{2} ( t ) \\ ⋮ \\ {S}_{n} ( t )\end{matrix} )}^{\\ {Component Signal}^{Independent}} & ( 2 )\end{matrix}$$

As described above, the photobiological measuring device according to the present example can generate unwanted component removal observation signals Xₙ′(t) by removing the signals corresponding to an unwanted component from the observation signals Xₙ(t).

In addition, in the photobiological measuring device according to the present example, the above described predetermined frequency band may be at least one frequency band selected from the group consisting of a frequency band indicating the blood flow in the skin, a frequency band indicating fluctuations in the heart rate, and a frequency band indicating pulsation or respiration.

Furthermore, the analysis method according to the present invention is an analysis method for generating an unwanted component removal observation signal by removing the signal corresponding to an unwanted component from an observation signal using a photobiological measuring device having: a light transmitting/receiving unit having a number of light transmitting probes to be provided on the surface of the skin of a subject and a number of light receiving probes to be provided on the surface of the skin; and a light transmitting/receiving unit control unit for acquiring an observation signal indicating chronological variations concerning a portion to be measured in a subject by allowing the above described light transmitting probes to irradiate the surface of the skin with light while controlling the above described light receiving probes in order to detect light emitted from the surface of the skin, the analysis method having; a mixing matrix making step of separating observation signals into the products of a mixing matrix and independent component signals through independent component analysis; and a unwanted component signal determining step of finding the independent component signal corresponding to an unwanted component from among independent component signals by comparing the power spectrum in a predetermined frequency band in each transformed independent component signal with a threshold.

### BRIEF DESCRIPTION OF THE DRAWINGS

FIG. 1 is a block diagram showing the structure of the photobiological measuring device according to one embodiment of the present invention;

FIG. 2 is a plan diagram showing the positional relationships between 16 light transmitting probes and 16 light receiving probes in a holder;

FIG. 3 is a diagram showing a monitor screen displayed on the photobiological measuring device according to the present invention;

FIG. 4 is another diagram showing a monitor screen displayed on the photobiological measuring device according to the present invention; and

FIG. 5 is still another diagram showing a monitor screen displayed on the photobiological measuring device according to the present invention.

### DETAILED DESCRIPTION OF THE INVENTION

In the following, the embodiments of the present invention are described in reference to the drawings. Here, the present invention is not limited to the following embodiments, but of course includes various modifications as long as the gist of the present invention is not deviated from.

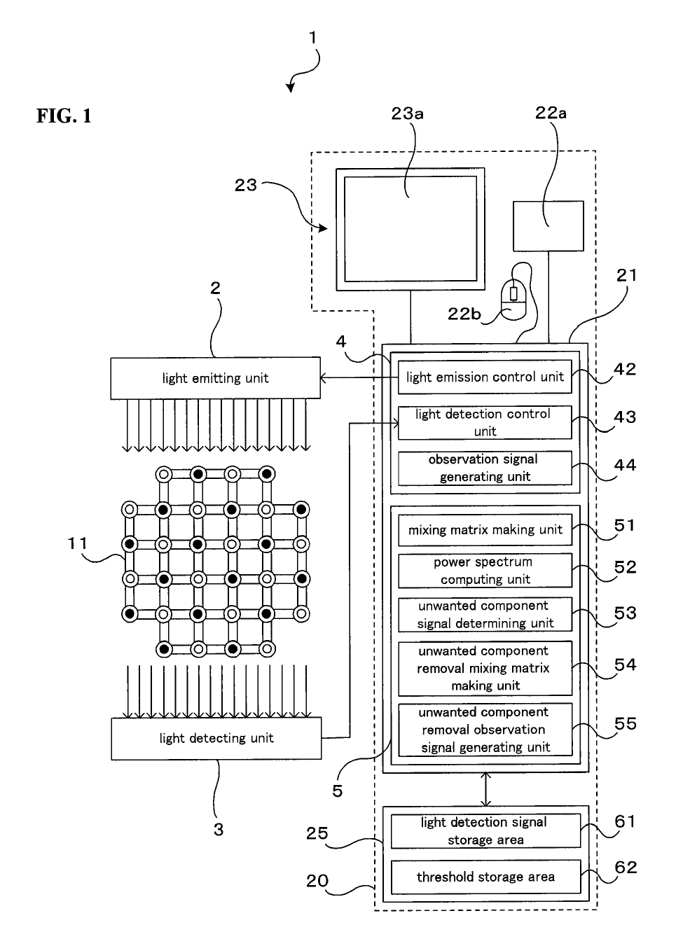

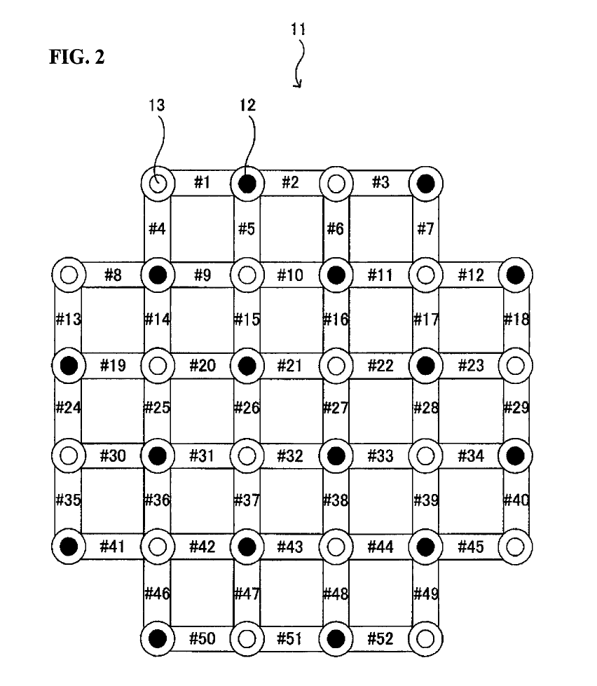

FIG. 1 is a block diagram showing the structure of the photobiological measuring device according to one embodiment of the present invention. In addition, FIG. 2 is a plan diagram showing the positional relationships between 16 light transmitting probes and 16 light receiving probes in a holder (light transmitting/receiving unit).

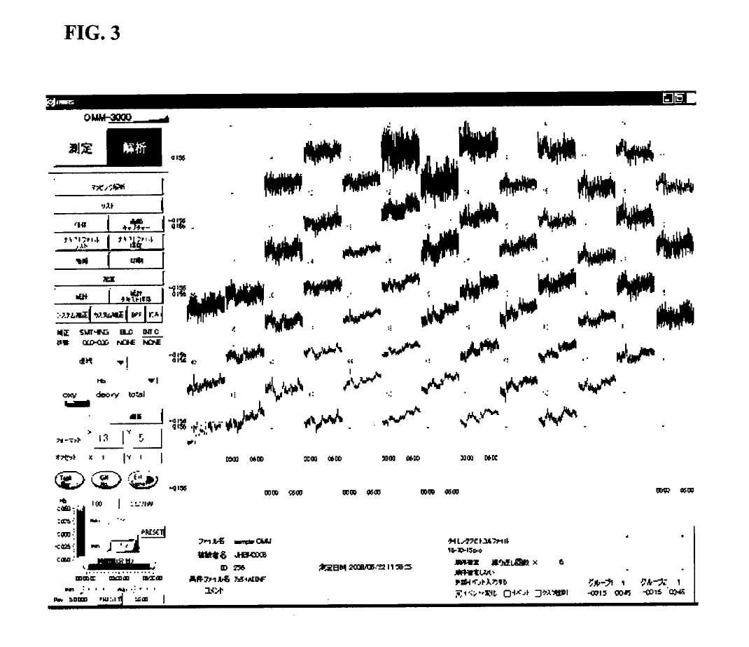

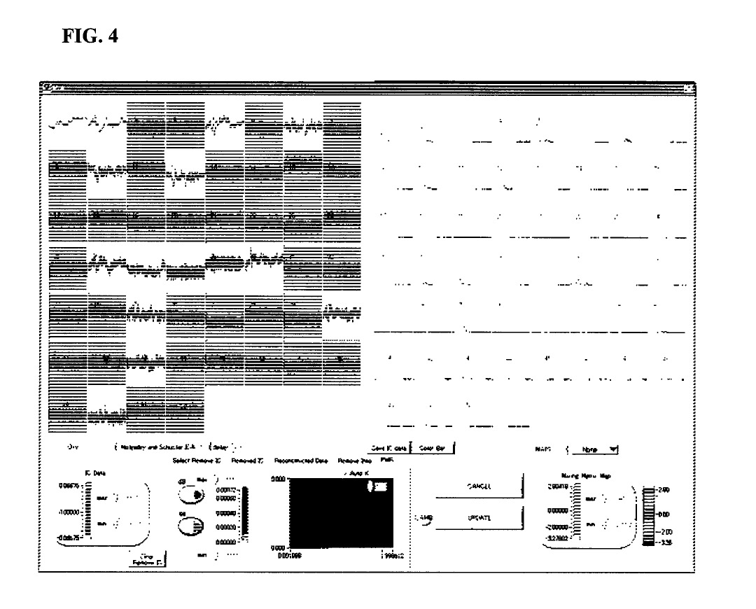

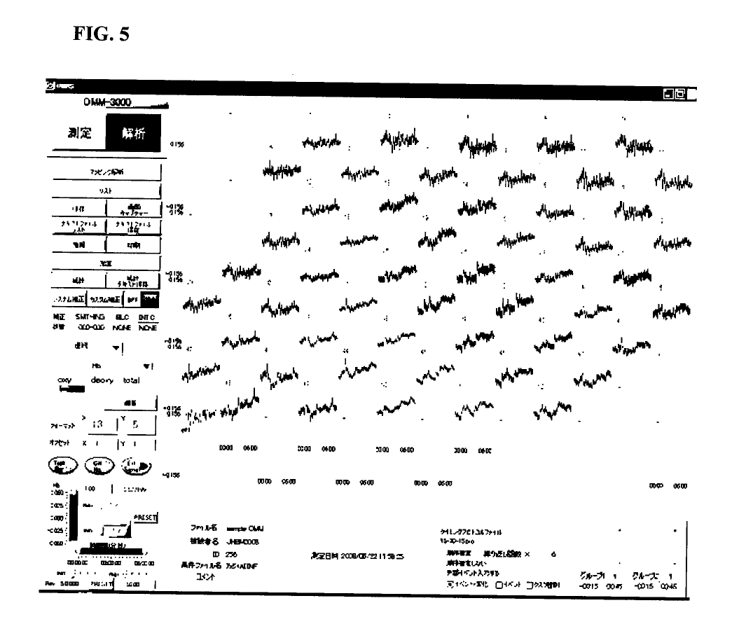

Furthermore, FIGS. 3 to 5 are diagrams showing a monitor screen displayed on the photobiological measuring device according to the present invention. FIG. 3 shows a monitor screen of the photobiological measuring device displaying 52 observation signals Xₙ(t), FIG. 4 shows a monitor screen of the photobiological measuring device displaying 52 independent component signals Sₙ(t) and the transformed independent component signals Sₙ(λ), and FIG. 5 shows a monitor screen of the photobiological measuring device displaying 52 unwanted component removal observation signals Xₙ′(t). The photobiological measuring device 1 is formed of a holder 11, a light emitting unit 2, a light detecting unit 3 and a control unit (computer) 20 for controlling the entirety of the photobiological measuring device 1.

As shown in FIG. 2, the holder 11 has 16 light transmitting probes 12 and 16 light receiving probes 13 in such a manner that the light transmitting probes 12 and the light receiving probes 13 are arranged alternately in the longitudinal and lateral directions. Here, the distance between the light transmitting probes 12 and the light receiving probes 13 is 30 mm. In addition, the 16 light transmitting probes 12 emit light while the 16 light receiving probes 13 detect the amount of light (light detection signal).

The light emitting unit 2 transmits light to one light transmitting probe 12 selected from among the 16 light transmitting probes 12 by the drive signal inputted from the computer 20. The above described light is near-infrared rays (for example, 780 nm and 850 nm).

The light detecting unit 3 individually detects near-infrared rays (for example, 780 nm and 850 nm) received by the 16 light receiving probes 13, and thus outputs the 16 light detection signals to the computer 20. The computer 20 is provided with a CPU 21 and connects to a memory 25, a display 23 having a monitor screen 23a, and a keyboard 22a and a mouse 22b, which are input devices 22.

For the sake of description, the functions processed by the CPU 21 can be divided into blocks: a light transmitting/receiving unit control unit 4 for acquiring observation signals Xₙ(t) by controlling the light emitting unit 2 and the light detecting unit 3; and a computing unit 5 for generating unwanted component removal observation signals Xₙ′(t). Furthermore, the computing unit 5 has a mixing matrix making unit 51, a power spectrum computing unit 52, an unwanted component signal determining unit 53, an unwanted component removal mixing matrix making unit 54 and an unwanted component removal observation signal generating unit 55.

Furthermore, the memory 25 has a light detection signal storage area 61 for storing light detection signals and a threshold storage area 62 for storing predetermined frequency bands λ₁ and thresholds T.

The light transmitting/receiving unit control unit 4 has a light emission control unit 42 for outputting a drive signal to the light emitting unit 2, a light detection control unit 43 for storing a light detection signal in the light detection signal storage area 61 when the light detection signal is inputted from the light detecting unit 3, and an observation signal generating unit 44 for generating observation signals Xₙ(t) which indicate chronological variations in the concentration of oxygenated hemoglobin.

The light emission control unit 42 controls the drive signal for transmitting light to the light transmitting probes 12 so that the drive signal is outputted to the light emitting unit 2.

The light detection control unit 43 controls 16 light detection signals detected from the 16 light receiving probes 13 so that the light detection signals are stored in the light detection signal storage area 61 when a light detection signal is inputted from the light detecting unit 3. That is to say, whenever light is transmitted from one light transmitting probe 12, 16 light detection signals are stored in the light detection signal storage area 61.

The observation signal generating unit 44 acquires 52 light detection signals, which are stored in the light detection signal storage area 61 and transmitted from a light transmitting probe 12 to the adjacent light receiving probes 13, and generates observation signals Xₙ(t) indicating chronological variations in the concentration of oxygenated hemoglobin on the basis of the acquired 52 light detection signals.

That is to say, the light detection signals for light from a light transmitting probe 12 to adjacent light receiving probes 13 are regarded as light detection signals gained from portions of the brain to be measured #1 to #52, and therefore after light is transmitted from all the light transmitting probes 12, 52 light detection signals selected from 256 light detection signals are gained. Then, on the basis of the thus-gained 52 light detection signals, the concentrations of oxyhemoglobin (observation signals) Xₙ(t) in the 52 portions on the surface of the brain (#1 to #52) are found (n=1, 2 . . . 52). As a result, as shown in FIG. 3, 52 observation signals Xₙ(t) (#1 to #52) can be gained.

As shown in the formula (1), the mixing matrix making unit 51 separates the 52 observation signals Xₙ(t) into the products of a 52×52 mixing matrix and 52 independent component signals Sₙ(t) through independent component analysis (mixing matrix making step).

The power spectrum calculating unit 52 Fourier transforms 52 independent component signals Sₙ(t), which are functions of time and intensity, so as to generate 52 transformed independent component signals Sₙ(λ), which are functions of frequency and intensity, and thus calculates the respective power spectra of a predetermined frequency band λ₁ in each transformed independent component signal Sₙ(λ). As a result, as shown in FIG. 4, 52 independent component signals Sₙ(t) and transformed independent component signals Sₙ(λ) are gained (#1 to #52).

The unwanted component signal determining unit 53 compares the power spectrum in the predetermined frequency band λ₁ in each transformed independent component signal Sₙ(λ) with the threshold T, respectively, and finds the independent component signal Sₙ(t) corresponding to an unwanted component from among the 52 independent component signals Sₙ(t) (unwanted component signal determining step). When the power spectrum S₁(λ₁) is not less than the threshold T, for example, the independent component signal S₁(t) is determined to be the signal corresponding to an unwanted component. Meanwhile, when the power spectrum S₁(λ₁) is less than the threshold T, the independent component signal S₁(t) is determined to be a signal on the basis of the blood flow according to the brain function. In addition, when the power spectrum S₂(λ₂) is not less than the threshold T, the independent component signal S₂(t) is determined to be the signal corresponding to an unwanted component. Meanwhile, when the power spectrum S₂(λ₂) is less than the threshold T, the independent component signal S₂(t) is determined to be a signal on the basis of the blood flow according to the brain function. Thus, the independent component signals Sₙ(t) corresponding to the unwanted components are found from among the 52 independent component signals Sₙ(t).

The unwanted component removal mixing matrix making unit 54 makes an unwanted component removal mixing matrix by substituting zero for the column vector corresponding to the unwanted component in the mixing matrix on the basis of the independent component signals Sₙ(t) corresponding to the unwanted components found by the unwanted component signal determining unit 53. When the power spectrum S₁(λ₁) is not less than the threshold T, for example, an unwanted component removal mixing matrix is made by substituting 0 for the first column vector. In addition, when the power spectrum S₂(λ₁) is not less than the threshold T, an unwanted component removal mixing matrix is made by substituting 0 for the second column vector. Thus, an unwanted component removal mixing matrix is made.

As shown in the formula (2), the unwanted component removal observation signal generating unit 55 multiplies the unwanted component removal mixing matrix by 52 independent component signals Sₙ(t) in order to generate 52 unwanted component removal observation signals Xₙ′(t). As a result, as shown in FIG. 5, 52 unwanted component removal observation signals Xₙ′(t) (#1 to #52) are gained.

As described above, the photobiological measuring device 1 can generate unwanted component removal observation signals Xₙ′(t) by removing signals corresponding to an unwanted component from the observation signals Xₙ(t). Accordingly, the observer, such as a doctor, can observe the unwanted component removal observation signals Xₙ′(t) in order to easily diagnose whether or not there is a condition such as cerebral ischemia.

### Other Embodiments

Though a holder 11 having 16 light transmitting probes 12 and 16 light receiving probes 13 is provided in the above described photobiological measuring device 1, the holder may have a different number of light emitting probes and light receiving probes, for example, 12 light emitting probes and 12 light receiving probes.

The present invention can be applied to an optical encephalographic device for measuring the function of a portion of the brain in a non-invasive manner using near-infrared rays (fNIRS: functional near-infrared spectroscope), or an oxygen monitor for monitoring the amount of oxygen consumption in the portion to be measured in a living body.

1. A photobiological measuring device, comprising:
a light transmitting/receiving unit having a number of light transmitting probes to be provided on the surface of the skin of a subject and a number of light receiving probes to be provided on the surface of the skin;
a light transmitting/receiving unit control unit acquiring an observation signal indicating chronological variations concerning a portion to be measured in a subject by allowing said light transmitting probes to irradiate the surface of the skin with light while controlling said light receiving probes in order to detect light emitted from the surface of the skin; and
a computing unit generating an unwanted component removal observation signal by removing the signal corresponding to the unwanted component from said observation signal, wherein
said computing unit comprises:
a mixing matrix making unit separating observation signals into the products of a mixing matrix and independent component signals through independent component analysis;
a power spectrum computing unit generating transformed independent component signals, which are functions of frequency and intensity, by Fourier transforming the independent component signals, which are functions of time and intensity, and thus computing power spectra in predetermined frequency bands of the transformed independent component signals; and
an unwanted component signal determining unit detecting the independent component signal corresponding to the unwanted component from among the independent component signals by comparing the power spectrum in a predetermined frequency band of each transformed independent component signal with a threshold,

2. The photobiological measuring device according to claim 1, wherein said computing unit comprises:
an unwanted component removal mixing matrix making unit making an unwanted component removal mixing matrix in which 0 is substituted for the column vector corresponding to an unwanted component in said mixing matrix on the basis of the independent component signal corresponding to an unwanted component found in said unwanted component signal determining unit; and
an unwanted component removal observation signal generating unit generating unwanted component removal observation signals by multiplying an unwanted component removal mixing matrix with independent component signals.

3. The photobiological measuring device according to claim 1, wherein said predetermined frequency band is at least one frequency band selected from the group consisting of a frequency band indicating the blood flow in the skin, a frequency band indicating fluctuations in the heart rate, and a frequency band indicating pulsation or respiration.

4. An analysis method for generating an unwanted component removal observation signal by removing the signal corresponding to an unwanted component from an observation signal using a photobiological measuring device comprising:
a light transmitting/receiving unit having a number of light transmitting probes to be provided on the surface of the skin of a subject and a number of light receiving probes to be provided on the surface of the skin; and
a light transmitting/receiving unit control unit acquiring an observation signal indicating chronological variations concerning a portion to be measured in a subject by allowing said light transmitting probes to irradiate the surface of the skin with light while controlling said light receiving probes in order to detect light emitted from the surface of the skin,
the analysis method being characterized by comprising:
a mixing matrix making step of separating observation signals into the products of a mixing matrix and independent component signals through independent component analysis; and
a unwanted component signal determining step of finding the independent component signal corresponding to an unwanted component from among independent component signals by comparing the power spectrum in a predetermined frequency band in each transformed independent component signal with a threshold.
